(19) United States
(12) Patent Application Publication
(10) Pub. No.: US 2020/0093703 A1
(43) Pub. Date: Mar. 26, 2020

(54) MEDICATION HANDLING SAFETY SYSTEM

(71) Applicants: Elie Fried, Flushing, NY (US); Paul S. Fried, Brooklyn, NY (US)

(72) Inventors: Elie Fried, Flushing, NY (US); Paul S. Fried, Brooklyn, NY (US)

(21) Appl. No.: 16/579,449

(22) Filed: Sep. 23, 2019

Related U.S. Application Data
(60) Provisional application No. 62/737,023, filed on Sep. 26, 2018.

(51) Int. Cl. A61J 7/00; G01J 3/02; A61J 1/03; G02B 26/00

(52) U.S. Cl. CPC A61J 7/0076; G01J 3/0254; G16H 20/13; G02B 26/008; A61J 2205/20; A61J 1/03

(57) ABSTRACT

A method for reducing drug administration errors is provided. The method may include manufacturing a drug with a specified color indicator. The method may include receiving an instruction to dispense a desired medication and dosage. The method may include loading a drug into a dispensing device. The method may include initiating a color spectroscopy sequence. The method may include determining, using color spectroscopy, the class of the loaded drug. The method may include determining whether the class of the loaded drug matches the class of the desired medication and dosage.

### BACKGROUND

The invention relates generally to a method and system for reducing medical errors, and more particularly, to a method and system for proper drug identification and minimizing errors in prescribing and dispensing medication.

Hospitals and medical centers dispense millions of medications (referred to herein as “drugs”) across the United States each day. With thousands of unique variations of drugs in existence, whole medical departments are devoted to dispensing and managing such drugs.

Errors in dispensing prescriptions are well-documented, and their existence is widespread. Some estimates assume a 2-3% rate of error in medication prescription or dispensing/administration, with tens of millions of prescriptions and drugs erroneously administered each year.

While some errors are harmless or relatively minor, others may be serious or fatal. This is particularly true in the operating room setting, where most prescriptions are carefully dosed based on a patient's current state or condition. While many medical errors have been found to be preventable, a reliable system in the operating room for reducing occurrences of drug mislabeling, erroneous dosing, and under- or over-dosing does not yet exist.

Current systems utilize color differentiation to indicate a type or category of drug in the operating room. For example, anesthesiologists retrieve a drug from a medical cart, ascertain the medication from the vial or ampule label, draw the medication for delivery, and then label the syringe with a color coded adhesive. The adhesive may also be marked with additional suitable information, such as the name of the drug, concentration, and date/time drawn, or any other suitable information.

While the American Society for Testing and Materials (ASTM) and International Organization for Standardization (ISO) have established standardized colors for drug categories, current categories fall short of significantly reducing or eliminating label-related errors. Labeling of a syringe of drugs, in the current standard, is implemented after a syringe is packed. Thus, the drug itself (the liquid) is not labeled, and instead, only the outer packaging is labeled. This fails to address the possibility of mislabeling the syringe contents on the outside of the syringe, and is not fail-safe.

It would be desirable therefore to provide a system and method for properly identifying drugs in the operating theater. It would be further desirable to provide a system and method for reducing errors, while indicating and ensuring proper medication labeling.

It would be further desirable to provide systems and methods for properly identifying and labeling the contents of a drug itself.

It would be yet further desirable provide a safety check system before loading onsite drug infusion systems.

It would be yet further desirable to provide a safety mechanism for remote automated delivery systems.

Such systems and methods for increasing drug safety and reducing error are hereby provided.

### SUMMARY OF THE INVENTION

A method for reducing drug administration errors is provided. The method may include manufacturing a drug with a specified color indicator. The method may include receiving an instruction to dispense a desired medication and dosage. The method may include loading a drug into a dispensing device. The method may include initiating a color spectroscopy sequence. The method may include determining, using color spectroscopy, the class of the loaded drug. The method may include determining whether the class of the loaded drug matches the class of the desired medication and dosage.

### BRIEF DESCRIPTION OF THE DRAWINGS

Non-limiting and non-exhaustive embodiments of the present embodiments are described with reference to the following FIGURES, wherein like reference numerals refer to like parts throughout the various views unless otherwise specified.

FIG. 1 depicts a method in accordance with an embodiment.

FIG. 2 depicts a method in accordance with a further embodiment.

### DETAILED DESCRIPTION OF THE INVENTION

Disclosed herein is a system and method for drug administration with reduced or eliminated drug selection errors.

In an embodiment, the invention includes a drug. The drug may be any suitable drug or pharmaceutical. The drug may be used as an anesthetic, or as part of an anesthetic cocktail. Exemplary drugs may include, but are not limited to opioid narcotics, paralytics, vasoconstrictors, or any other suitable drugs.

In an embodiment, an identifying color may be mixed with the drug.

In an embodiment, the system may be used for gaseous drugs, foams, liquid drugs, or any other suitable form. In an embodiment, they system may be utilized with a gaseous drug undergoing vaporization.

The system disclosed herein is specifically formed to address a possibility of drug substitution and/or mislabeling. As described, the invention avoids the need to use costly pre-filled syringes, while allowing for the proper labeling of medication with name, date and concentration. The system also allows for using pre-printed, color-coded, and bar-coded labels.

In an embodiment, the system and method include “labeling” of a drug at the manufacturing or bottling stage. The drug is labeled by coloring the drug, such as an anaesthetic. For example, as the drug may be manufactured and, before bottling, coloring is introduced into the drug mixture. In a further example, a specific color is chosen, indicating the specific type of drug.

Exemplary embodiments of color coding may include, but are not limited to, utilizing a blue color liquid for opioids or narcotics, red for paralytic agents, purple for vasopressor agents, and green for anticholinergic agents. For example, blue may be a methylene blue solution, and green may be an indocyanine green solution.

In an embodiment, a color additive to the drug solution may be used. The color additive may be chemically inert.

In one embodiment, prior to administration, a drug vial or syringe may be placed in a holder. The holder may include a sensor. The holder may include a spectrometer. The sensor may detect a color range of the liquid drug within the vial or syringe. Upon detection, the sensor may then determine, for example, based on a specific shade, the exact drug within the vial. The sensor may then instruct the holder to administer the drug. In another embodiment, the system may be provided with a desired drug. The system may compare the drug in the holder, determined by the color, to the desired drug. If the desired drug and the determined drug do not match, the system may set a flag. The system may further prevent the drug from administration. In a further embodiment, the system may determine that the color of the drug is not the appropriate color. The system may then flag the vial containing the drug for further review.

In an embodiment, color spectroscopy may be used. Color spectroscopy may be used to identify a solution through its color signature. That is, the color signature may be detected and determined. In another embodiment, the device may incorporate a spectrophotometer unit. The unity may be programmed to determine whether a specific color wavelength is present. For example, the unit may be set to determine whether a wavelength of 450 nm is present, corresponding to a specific hue of blue colored solution in that range. The unit may then provide an indication of whether or not the programmed color solution is present.

In a further embodiment, blue may be in the 450-495 nm wavelength, and red in the 620-750 nm wavelength, and can be set to determine whether an unknown solution is one of the two colors. Additionally, the unit may be programmed to take a further action on determination that a specified color, such as 450 nm blue, is present (e.g., cause an IV pump to flow, or set an alarm, or stop an IV pump).

The color spectroscopy may be incorporated into one or more drug infusion systems. For example, using a color spectroscopy-enabled system, identification of a drug to be administered may be ascertained based on the drug color. The drug color may be matched against an approved color. If the drug color is determined to be correct, the system may allow for drug infusion to proceed. If the drug color is determined to be improper or incorrect, the system may initiate an alarm, an error sequence, or may lock the administration system for a predetermined period of time.

Thus, in an embodiment, a camera may capture images of a vial. The camera may capture an image of the drug within the vial.

In accordance with the invention, the system reduces human contributions to medication-administered errors, and specifically, those in the operating environment. Utilizing a color-coded liquid, risks of administering an improper drug, resulting in drug substitution, is drastically reduced.

In accordance with an embodiment, spectroscopy may be used to determine a color intensity, in order to differentiate between drugs of a same class. For example, one shade of a color, such as blue, may be used to label Fentanyl, while another shade of blue may be used to code another drug in the same class, such as Morphine. Spectroscopy may then be used to determine the specific drug.

In another embodiment, spectroscopy may be used to differentiate between different concentrations of a same drug, such as, for example, deep purple for 1 mg/cc of Epinephrine, while a lighter purple may be coded to 0.1 mg/cc of Epinephrine. In an embodiment, the shade (such as a lighter or darker shade) may correspond to the concentration, so a darker shade corresponds to a higher concentration than a lighter shade.

In accordance with an embodiment, the use of regulated and centralized pharmaceutical manufacturing processes allows for coloring of a drug with the correct color, resulting in a reduction in risk of mis-coloring (and thereby reducing mis-labeling).

It should be noted that in an embodiment, a drug is color-coded by class. Thus, if the wrong drug is used, but of the same class, the risk of impact on patient safety is significantly reduced, as compared to a drug switch involving a different drug class. For example, administration of a different narcotic/opioid in place of the intended one is less likely to result in significant patient harm, when compared to the administration of a paralytic agent in place of a narcotic/opioid.

In a further embodiment, common drugs may be color coded with a specific color, that is coded only to that particular drug. For example, the color may be coded a specific shade of blue for one drug in a class, and a second shade of blue for a second drug in that same class. This embodiment may optionally be used with color spectroscopy systems as discussed above, such that minute changes in color, which are not objectively perceptible to the human eye, can be readily detected by such a system to reduce error.

An advantage of the disclosed system allows for use of color as a visual cue, which is more quickly identified than written information. Coloring of a drug also avoids problems associated with a label being blocked, or other markings being obscured.

The system, in accordance with an embodiment, contemplates use in tele-anesthesia settings. Utilizing artificial intelligence, neural networks, and deep learning generated algorithms, automated systems are configured to perform automated anesthesia care in complex environments. Through the use of intravenous drugs of different colors, safety can be improved and error reduced. Such a system, when incorporated into tele-anesthesia, would allow for the use of onsite, minimally or lesser trained technicians to set up drug administration systems, for tele-anesthesia control by a remote anesthesiologist. The color coding of various drugs would provide an added level of safety.

In an exemplary embodiment, color spectroscopy may be used to determine the use of a proper drug, and prevent administration of an improper drug. Thus, an anesthesia system may receive an instruction to dispense a drug. A drug may then be loaded into the system. Color spectroscopy may be then be used to determine the color of the loaded drug. Based on the color, the system may then determine whether the loaded drug is the same class, or specific drug, as that of the instructed drug. In an embodiment, the system may be determined for a specific drug, or for a specific class of drug. In a further embodiment, the system may initiate an error sequence when the wrong drug loaded, or alternatively, only when a different drug class is loaded.

Exemplary color spectroscopies may be violet, at 400-420 nm, blue, at 440-490 nm, green, at 490-570 nm, yellow, at 570-585 nm, and red, at 620-780 nm.

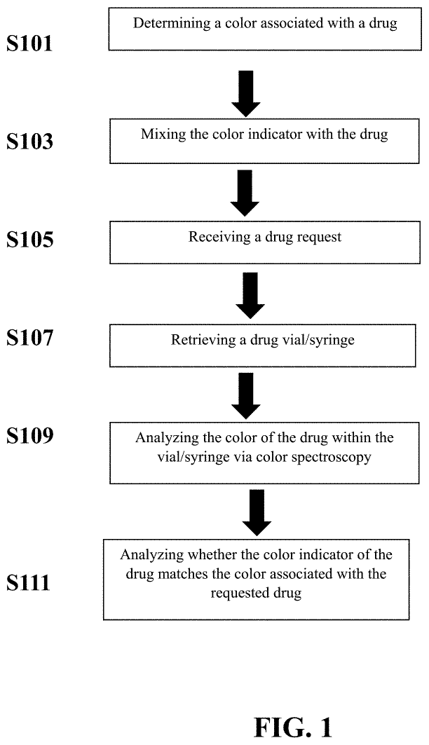

Referring now to the Figures, FIG. 1 illustrates an exemplary process in accordance with an embodiment. In an embodiment, a drug or plurality of drugs may be classified into a class. At S101, the system may determine a color associated with a drug, or with a drug class. At S103, the system may mix a color indicator, of the same color determined in S101, with the drug. At S105, the system may receive a drug request, such as a drug request from a medical professional. At S107, the system may retrieve a drug vial or syringe. At S109, the system may analyze the color of the drug within the drug vial or syringe. The color may be analyzed via color spectroscopy. At S111, the system may analyze whether the color indicator of the drug matches the color associated with the requested drug.

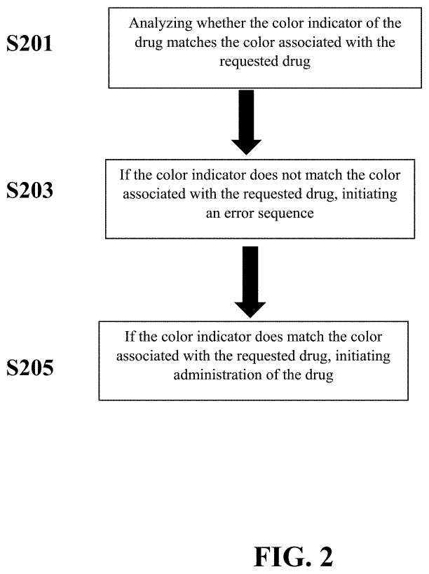

Referring now to FIG. 2, shown is an exemplary process once the color indicator is analyzed. S201 may be identical to S111. At S203, the system may initiate an error sequence if the color indicator does not match the color associated with the requested drug. The error sequence may include preventing administration of the drug. In an embodiment, administration of the drug may be prevented by preventing the drug holder or dispensing device from administering the drug. At S205, the system may initiate administration of the drug if the color of the drug matches the color of the desired indicator.

Therefore, in accordance with an embodiment, the system may be utilized in a clinical setting. An IV infusion pump may be loaded with a syringe containing medication for delivery. In one embodiment, opioids may be blue, whereas paralytics may be red. The infusion pump may include a spectrophotometer, and may be programmed to measure color, and only cause an action if the preset color is found in the loaded syringe. For example, the pump may be programmed to only deliver medication that measures red, and may be programmed to take an action (such as setting an alarm or cutting off flow) when a medication with the improper color is detected.

In an embodiment, a system in accordance with an embodiment may be programmed to measure the concentration of a solution. Thus, the absorbance of a solution is proportional to the concentration of the solution, and therefore, the color of the solution. The system may therefore determine absorbance of a solution, thereby determining a concentration. In accordance with an embodiment, different hues/shades of color within a specific drug class (such as dark blue for fentanyl or light blue for morphine) may be used to indicate different drugs within a same class. This overcomes a deficiency in manual visual review, which is not able to detect minute changes in hue and/or wavelength. That is, in conventional methods, human eyes are only able to detect a differentiation on a broader level, rather than identifying numerous drugs of different specific hues within the same class. Therefore, medical administration errors are reduced.

In accordance with an embodiment, them system may provide for reduction of errors relating to mixing drug classes. That is, in differentiating between colors, the system may allow for properly identifying the class of drug, based on the color of the drug solution. The system may also improve the ability to differentiate hues or saturations of the same color, allowing for detection of different drugs within the same class. A digital library of acceptable shades may be compiled, and the system may analyze and compare a solution against the spectrum of acceptable shades. This may either then prevent an action (such as flow), or may allow an action.

In one embodiment, the system may be used during drawing up and preparing a drug for administration, and labeling a syringe, prior to a procedure.

Thus, in accordance with the systems and methods disclosed herein avoid errors arising from practitioners erroneously printing syringe labels, or the cost of preloaded syringes.

Utilizing such systems and methods with infusion devices provides for a system that reduces or eliminates errors, or prevents functioning, when a mismatch between a drug to be delivered and the actual selected drug occurs.

What is claimed is:

1) A method for reducing drug administration errors, comprising:
manufacturing a drug with a specified color indicator;
receiving an instruction to dispense a desired medication and dosage;
loading a drug into a dispensing device;
initiating a color spectroscopy sequence;
determining, using color spectroscopy, the class of the loaded drug;
determining whether the class of the loaded drug matches the class of the desired medication and dosage.

2) The method of claim 1, further comprising, where the class of the loaded drug does not match the class of the desired medication and dosage, initiating an error sequence.

3) The method of claim 2, wherein the error sequence prevents administration of the drug.

4) The method of claim 1, further comprising where the class of the loaded drug matches the class of the desired medication and dosage, administering the drug.

5) A system for reducing drug administration errors, comprising:
a drug manufacturing component;
a receiver configured to receive an instruction to dispense a desired medication;
a dispensing device;
a loader for the loading a drug into the dispensing device; and
a color spectrophotometer.

6) The system of claim 5, wherein the loader is configured to load the drug into the dispensing device.

7) The system of claim 6, wherein the color spectrophotometer is configured to initiate a color identification sequence for the drug.

8) The system of claim 7, wherein the color identification sequence includes determining a class of the loaded drug based on the identified color.

9) The system of claim 8, further comprising determining whether the class of the loaded drug matches the drug received in the instruction to dispense the desired medication.

10) The system of claim 9, wherein the drug is prevented from administration until confirmation that the loaded drug is identified, via the spectrophotometer, as a correctly colored drug.
